(19) United States
(12) Patent Application Publication
(10) Pub. No.: US 2018/0098997 A1
(43) Pub. Date: Apr. 12, 2018

(54) PHARMACEUTICAL DOSES FOR A BROMODOMAIN AND EXTRATERMINAL PROTEIN (BET) INHIBITOR

(71) Applicant: Oncoethix GmbH, Lucerne (CH)

(72) Inventors: Esteban Cvitkovic, Paris (FR); Patrice Herait, Soisy-sous-Montmoreacy (FR)

(73) Assignee: Oncoethix GmbH, Lucerne (CH)

(21) Appl. No.: 15/561,323

(22) PCT Filed: Apr. 3, 2015
(86) PCT No.: PCT/IB2015/000624

§ 371 Date: Sep. 25, 2017

(51) Int. Cl. A61K 31/551; A61P 35/00; A61P 35/02

(52) U.S. Cl. CPC A61K 31/551; A61P 35/02; A61P 35/00

(57) ABSTRACT

The invention is a method for treating patients with cancer comprising administering to the patient a safe and effective dose of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamideor a pharmaceutically acceptable salt or hydrate thereof, wherein the dose is between about 40 mg once per per day and about 120 mg once per per day. In one embodiment of the invention, the dose is about 40 mg once per per day, about 80 mg once per day or about 120 mg once per per day. In another embodiment of the invention, the dose is about 40 mg once per day. In another embodiment of the invention, the dose is about 80 mg once per day. In another embodiment of the invention, the dose is about 120 mg once per day. In another embodiment of the invention, the cancer is acute leukemia, older adult hematologic malignancy, lymphoma or multiple myeloma. In another embodiment of the invention, the lymphoma is diffuse large B-cell lymphoma.

### BACKGROUND OF THE INVENTION

The compound of Formula (1)

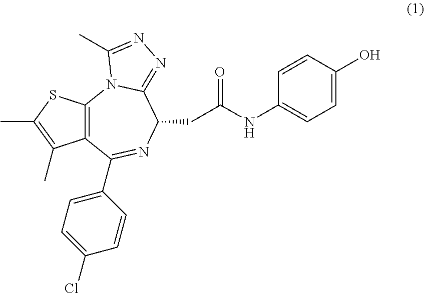 [SMILES: Cc1sc2c(c1C)C(c1ccc(Cl)cc1)=N[C@@H](CC(=O)Nc1ccc(O)cc1)c1nnc(C)n1-2]

(S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide, has been shown to inhibit the binding of acetylated histone H4 to the tandem bromodomain (BRD)-containing family of transcriptional regulators known as the BET (bromodomains and extraterminal) proteins, which include BRD2, BRD3, and BRD4. See U.S. Patent Application Publication No. 2010/0286127 A1, which is incorporated herein by reference in its entirety. The compound displays in vitro and in vivo activity in a variety of hematologic and solid tumor preclinical models. The BET proteins have emerged as major epigenetic regulators of proliferation and differentiation and also have been associated with predisposition to dyslipidemia or improper regulation of adipogenesis, elevated inflammatory profile and risk for cardiovascular disease and type 2 diabetes, and increased susceptibility to autoimmune diseases such as rheumatoid arthritis and systemic lupus erythematosus as reported by Denis, G. V. “Bromodomain coactivators in cancer, obesity, type 2 diabetes, and inflammation,” Discov Med 2010; 10:489-499, which is incorporated herein by reference in its entirety.

Dosing of the compound of Formula (1) is described in Odore, et al., AACR Apr. 18-22, 2015 Annual Meeting Abstract 4511 entitled “Pharmacokinetics of OTX015 in a phase Ib dose-finding study of patients with hematologic malignancies: Preliminary results of a population PK analysis” which identifies once-a-day doses of 10, 20, 40, 80, 120 and 160 mg and twice-a-day doses of 40 mg. Clinical studies of the compound of Formula (1) are described on ClinicalTrials.gov: NCT02296476, “A Trial With Dose Optimization of OTX015 in Recurrent Glioblastoma Multiforme (GBM) Patients,” first received Oct. 3, 2014, which does not describe the doses studied; NCT01713582, “A Phase I, Dose-finding Study of the Bromodomain (Brd) Inhibitor OTX015 in Haematological Malignancies,” first received Oct. 22, 2012, which describes a 10 mg dose study; NCT02259114, “A Phase IB Trial With OTX015, a Small Molecule Inhibitor of the Bromodomain and Extra-Terminal (BET) Proteins, in Patients With Selected Advanced Solid Tumors,” first received Oct. 3, 2014, which does not describe the doses studied; and NCT02303782, “A Study Assessing OTX015 in Combination With Azacitidine (AZA) or AZA Single Agent in Patients With Newly-diagnosed Acute Myeloid Leukemia (AML) Not Candidate for Standard Intensive Induction Therapy (SIIT), first received Nov. 24, 2014, which does not describe the doses studied. Oncology Practice.com “Novel epigenetic treatment showed activity in hematologic cancers” Apr. 17, 2014 describes once-a-day doses of 10, 20, 40, and 80 mg and twice-a-day doses of 40 mg of the compound of Formula (1). ASCO Post.com

“Investigational Bromodomain Inhibitor Shows Clinical Activity in Some Blood Cancers” Apr. 8, 2014 describes once-a-day doses of 10, 40, 80, and 120 and twice-a-day doses of 40 mg of the compound of Formula (1). 56^{th} ASH Annual Meeting and Exposition San Francisco, Calif. Dec. 6-9, 2014 Abstract 117 “A Phase 1 Study of the BET-Bromodomain Inhibitor OTX015 in Patients with Advanced Acute Leukemia” describes once-a-day doses of 10, 20, 40, 80, 120 and 160 mg and twice-a-day doses of 40 mg of the compound of Formula (1).

In the description below, unless otherwise indicated, the phrase “compound of Formula (1)” includes (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide and pharmaceutically acceptable salts and hydrates thereof.

### SUMMARY OF THE INVENTION

The invention is a method for treating patients with cancer comprising administering to the patient a dose of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, wherein the dose provides safe and effective drug exposure. In one embodiment of the invention, the dose is between about 40 mg once per per day and about 120 mg once per per day. In another embodiment of the invention, the dose is selected from about 40 mg once per per day, about 80 mg once per day and about 120 mg once per per day. In another embodiment of the invention, the dose is about 40 mg once per day. In another embodiment of the invention, the dose is about 80 mg once per day. In another embodiment of the invention, the dose is about 120 mg once per day. In another embodiment of the invention, the cancer is acute leukemia, older adult hematologic malignancy, lymphoma or multiple myeloma. In another embodiment of the invention, the lymphoma is diffuse large B-cell lymphoma. In another embodiment of the invention, drug exposure corresponds to AUC₀₋₂₄ of between about 3000 to about 20000 μg/L*h. In another embodiment of the invention, drug exposure corresponds to AUC₀₋₂₄ of between about 5000 to about 18000 μg/L*h. In another embodiment of the invention, drug exposure corresponds to T_{1/2} of between about 5 and about 6 hours. In another embodiment of the invention, drug exposure corresponds to Cₘₐₓ of between about 500 and about 1500 μg/L. In another embodiment of the invention, drug exposure corresponds to Cₘₐₓ of between about 600 and about 1500 μg/L.

The invention is also a method for treating patients with cancer comprising administering to the patient a safe and effective dose of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, wherein the dose is between about 40 mg once per per day and about 120 mg once per per day. In one embodiment of the invention the dose is about 40 mg once per per day, about 80 mg once per day or about 120 mg once per per day. In another embodiment of the invention, the dose is about 40 mg once per day. In another embodiment of the invention, the dose is about 80 mg once per day. In another embodiment of the invention, the dose is about 120 mg once per day. In another embodiment of the invention, the cancer is acute leukemia, older adult hematologic malignancy, lymphoma or multiple myeloma. In another embodiment of the invention, the lymphoma is diffuse large B-cell lymphoma.

The invention is also a pharmaceutical composition comprising between 40 mg and 120 mg (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, and a pharmaceutically acceptable carrier. In one embodiment of the invention, the pharmaceutical composition comprises about 40 mg (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, and a pharmaceutically acceptable carrier. In another embodiment of the invention the pharmaceutical composition comprises about 80 mg (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, and a pharmaceutically acceptable carrier. In one embodiment of the invention, the pharmaceutical composition comprises about 120 mg (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, and a pharmaceutically acceptable carrier.

### DETAILED DESCRIPTION OF THE INVENTION

“Safe and effective” dosing to patients is dosing that induces an objective response in a patient while avoiding or minimizing undesirable side effects.

With regard to the compound of Formula (1), an effective dose is a dose that inhibits a protein in the bromodomain and extraterminal protein family to a sufficient degree that a desired objective response is observed. An objective response may be a complete response (all detectable tumor or blast cell has disappeared), a partial response (roughly corresponds to at least a 50% decrease in the total tumor volume or blast cell amount but with evidence of some residual disease still remaining), or a minor response (roughly means a small amount of tumor shrinkage or blast cell reduction). Thus, an objective response can include a response where blast cell amount reduction is observed, or where tumor shrinkage is observed in the patient.

With regard to the compound of Formula (1), a safe dose is a dose that does not induce dose-limiting toxicity in a patient. Dose-limiting toxicity corresponds to side-effect toxicity such that the side-effects are severe enough to impose restrictions on, or to prevent administration of, the dose. Clinically significant toxicity includes but is not limited to intolerable levels in a patient of thrombocytopenia, neutropenia, fatigue, diarrhea, anemia, mucositis, and other side effects known to be associated with elevated levels of bromodomain and extraterminal protein inhibitors.

Cancer includes but are not limited to, adrenal cancer, acinic cell carcinoma, acoustic neuroma, acral lentigious melanoma, acrospiroma, astute leukemia such acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma; astrocytoma, atypical teratoid rhabdoid tumor, B-cell chronic lymphocytic leukemia, B-cell prolymphocyte leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma; chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in feta, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy such as older adult hematological malignancy, hepatoblastorna, lymphoma including diffuse large B-cell lymphoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, Leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor. In one embodiment of the invention, cancer includes acute leukemia, older adult hematologic malignancy, lymphoma or multiple myeloma. Lymphoma includes diffuse large B-cell lymphoma.

Doses may also be used to treat benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

“AUC₀₋₂₄”, a measure of drug exposure, corresponds to the area under the plasma drug concentration versus time curve over a 24 hour period, and is represented in units μg/L*h. “Half-life” (T_{1/2}), the amount of time required for drug plasma level concentration, representing the amount of drug in the body, to be reduced by 50%, is measured in hours. “Cₘₐₓ”, the maximum concentration that a drug achieves in a patient after the drug has been administrated, and prior to the administration of a second dose, is represented in units μg/L.

In one embodiment of the invention, safe and effective drug exposure corresponds to AUC₀₋₂₄ of between about 3000 to about 20000 μg/L*h. In another embodiment, safe and effective drug exposure corresponds to AUC₀₋₂₄ of between about 5000 to about 18000 μg/L*h. In another embodiment, safe and effective drug exposure corresponds to T_{1/2} of between about 5 and 6 hours. In another embodiment, safe and effective drug exposure corresponds to Cₘₐₓ of between about 500 and about 1500 μg/L. In another embodiment, safe and effective drug exposure corresponds to Cₘₐₓ of between about 600 and about 1500 μg/L. Additional embodiments of safe and effective drug exposure correspond to any combination of the above safe and effective embodiments.

The doses of the invention provide to the patients drug exposure and maximum drug concentration associated with safe and effective administration of the compound of Formula (1).

The compound of Formula (1), pharmaceutically acceptable salts, solvates, including hydrates, racemates, enantiomers isomers, and isotopically-labeled forms thereof, can be formulated as a solid dispersion with pharmaceutically acceptable polymers to provide an oral formulation that provides high absorption of the pharmaceutical ingredient into the circulation from the gastrointestinal tract for treatment of diseases other than inflammatory bowel diseases. Studies in both dogs and humans have confirmed high oral bioavailability of these solid dispersions compared with the Eudragit solid dispersion formulation previously developed for the treatment of inflammatory bowel disease.

The term “solid dispersion” as used herein refers to a group of solid products including at least two different components, generally a hydrophilic carrier and a hydrophobic drug, the compound of Formula (1). Based on the drug's molecular arrangement within the dispersion, six different types of solid dispersions can be distinguished. Commonly, solid dispersions are classified as simple eutectic mixtures, solid solutions, glass solutions and suspensions, or amorphous precipitations in a crystalline carrier. Moreover, certain combinations can be encountered; for example, in the same sample some molecules may be present in clusters while some are molecularly dispersed.

The compound of Formula (1) can be dispersed molecularly in amorphous particles (clusters). The compound of Formula (1) can be dispersed as crystalline particles. The carrier can be, for example, crystalline, or the carrier can be amorphous.

The compound of Formula (1) can be prepared in a pharmaceutical composition comprising a solid dispersion of the compound, or a pharmaceutically acceptable salt, a solvate, including a hydrate, a racemate, an enantiomer, an isomer, or an isotopically-labeled form thereof; and a pharmaceutically acceptable polymer. The pharmaceutically acceptable polymer can be hypromellose acetate succinate (also called hydroxypropylmethylcellulose acetate succinate or HPMCAS). The dispersion can be prepared in a compound to hydroxypropylmethylcellulose acetate succinate (HPMCAS) weight ratio of about 1:3 to about 1:1. In such a dispersion, at least some portion of the compound is homogeneously dispersed throughout the solid dispersion, or the compound is homogeneously dispersed throughout the solid dispersion. The solid dispersion can exhibit a single inflection for the glass transition temperature (Tg). The single Tg can occur between about 130° C. to about 140° C.

The compound of Formula (1) can be prepared in a pharmaceutical composition comprising a solid dispersion of the compound or a pharmaceutically acceptable salt, a solvate, including a hydrate, a racemate, an enantiomer, an isomer, or an isotopically-labeled form thereof in a pharmaceutically acceptable polymer, wherein the polymer is polyvinylpyrrolidone (also called povidone or PVP). The dispersion can be prepared in a compound to PVP weight ratio of about 1:3 to about 1:1. In such a dispersion, at least some portion of the compound is homogeneously dispersed throughout the solid dispersion, or the compound is homogeneously dispersed throughout the solid dispersion. The solid dispersion can exhibit a single inflection for the glass transition temperature (Tg). The single Tg can occur between about 175° C. to about 185° C.

The bioavailability of the compound of Formula (1) may be measured by the area under the curve (AUC) value obtained by plotting a serum or plasma concentration of the compound along the ordinate (Y-axis) against time along the abscissa (X-axis).

Suitable dosage forms that can be prepared with the solid dispersions of the compound of Formula (1) include, but are not limited to, capsules, tablets, mini-tablets, beads, beadlets, pellets, granules, granulates, and powder. Suitable dosage forms may be coated, for example using an enteric coating. Suitable coatings may comprise but are not limited to cellulose acetate phthalate, hydroxypropylmethylcellulose (HPMC), hydroxypropylmethylcellulose phthalate, a polymethylacrylic acid copolymer, or hydroxylpropylmethylcellulose acetate succinate (HPMCAS).

In one embodiment, the solid dispersions of the compound of Formula (1) may be formulated as tablets, caplets, or capsules. The solid dispersions of the compound of Formula (1) may also be formulated as mini-tablets or pour-into-mouth granules, or oral powders for reconstitution. The solid dispersions of the compound of Formula (1) are dispersed in a suitable diluent in combination with other excipients to give a ready-to-use suspension formulation. The solid dispersions of the compound of Formula (1) may be formulated for pediatric treatment.

In one embodiment, the pharmaceutical compositions of the compound of Formula (1) can be formulated for oral administration, e.g., the composition comprises a solid dispersion as described herein, comprising the compound of Formula (1) or a pharmaceutically acceptable salt, a solvate, including a hydrate, a racemate, an enantiomer, an isomer, or an isotopically-labeled form thereof; and a polymer carrier. The pharmaceutical composition can include one or more additives such as disintegrants, lubricants, glidants, binders, and fillers.

Examples of suitable pharmaceutically acceptable lubricants and pharmaceutically acceptable glidants for use with the pharmaceutical composition of the invention include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose, glyceryl behenate, stearic acid, hydrogenated castor oil, glyceryl monostearate, and sodium stearyl fumarate.

Examples of suitable pharmaceutically acceptable binders for use with the pharmaceutical composition of the invention include, but are not limited to starches, celluloses and derivatives thereof (e.g., microcrystalline cellulose (e.g., AVICEL PH from FMC), hydroxypropyl cellulose, hydroxyethyl cellulose, and hydroxylpropylmethylcellulose (HPMC, e.g., METHOCEL from Dow Chemical), sucrose, dextrose, corn syrup, polysaccharides, and gelatin.

Examples of suitable pharmaceutically acceptable fillers and pharmaceutically acceptable diluents for use with the pharmaceutical composition of the invention include, but are not limited to, confectioner's sugar, compressible sugar, dextrates, dextrin, dextrose, lactose, mannitol, microcrystalline cellulose (MCC), powdered cellulose, sorbitol, sucrose, trehalose and talc.

Excipients may serve more than one function in the pharmaceutical composition. For example, fillers or binders may also be disintegrants, glidants, anti-adherents, lubricants, sweeteners and the like.

The pharmaceutical compositions of the compound of Formula (1) may further include additives or ingredients, such as antioxidants (e.g., ascorbyl palmitate, butylated hydroxylanisole (BHA), butylated hydroxytoluene (BHT), alpha.-tocopherols, propyl gallate, and fumaric acid), antimicrobial agents, enzyme inhibitors, stabilizers (e.g., malonic acid), and/or preserving agents.

Generally, the pharmaceutical compositions of the compound of Formula (1) may be formulated into any suitable solid dosage form. The solid dispersions of the invention are compounded in unit dosage form, e.g., as a capsule, or tablet, or a multi-particulate system such as granules or granulates or a powder, for administration.

The pharmaceutical compositions of the invention can include a solid dispersion of the compound of Formula (1), according to the various embodiments of solid dispersions described herein, and hydroxypropylmethylcellulose acetate succinate (HPMCAS), wherein the compound is amorphous in the solid dispersion and has a compound to hydroxypropylmethylcellulose acetate succinate (HPMCAS), weight ratio of about 1:3 to about 1:1; about 45-50 wt. % of lactose monohydrate; about 35-40 wt. % of microcrystalline cellulose; about 4-6 wt. % of croscarmellose sodium; about 0.8-1.5 wt. % of colloidal silicon dioxide; and about 0.8-1.5 wt. % of magnesium stearate.

The dosage form used, e.g., in a capsule, tablet, mini-tablet, beads, beadlets, pellets, granules, granulates, or powder may be coated, for example using an enteric coating. Suitable coatings may comprise but are not limited to cellulose acetate phthalate, hydroxypropylmethylcellulose (HPMC), hydroxypropylmethylcellulose phthalate, a polymethylacrylic acid copolymer, or hydroxylpropylmethylcellulose acetate succinate (HPMCAS).

The compound of Formula (1) disclosed herein can exist as free base or as acid addition salt and can be obtained according to the procedures described in US Patent Application Publication No. 2010/0286127, incorporated by reference in its entirety herein. Individual enantiomers and diastereomers of the compound of Formula (1) can be prepared synthetically from commercially available starting materials that contain asymmetric or stereogenic centers, or by preparation of racemic mixtures followed by resolution methods well known to those of ordinary skill in the art.

Formulations of the compound of Formula (1) can be prepared by a solvent evaporation method. The solvent evaporation method comprises solubilization of the compound of Formula (1) in a volatile solvent that is subsequently evaporated. The volatile solvent may comprise one or more excipients. The one or more excipients include, but are not limited to, anti-sticking agents, inert fillers, surfactants wetting agents, pH modifiers and additives. The excipients may be dissolved or in suspended or swollen state in the volatile solvent.

Preparation of solid dispersions includes drying one or more excipients suspended in a volatile solvent. The drying includes vacuum drying, slow evaporation of the volatile solvent at low temperature, use of a rotary evaporator, spray-drying, spray granulation, freeze-drying, or use of supercritical fluids.

Spray drying preparations of a formulation of the compound of Formula (1) involves atomization of a suspension or a solution comprising the compound into small droplets, followed by rapid removal of solvent from the formulation. Preparation of a formulation involves spray granulation in which a solution or a suspension of the composition in a solvent is sprayed onto a suitable chemically and/or physically inert filler, such as lactose or mannitol. In one embodiment, spray granulation of the solution or the suspension of the composition is achieved via two-way or three-way nozzles.

The invention is illustrated in the following non-limiting examples.

### Example 1

Solid dispersions were prepared using the compound of Formula (1) and hydroxylpropylmethylcellulose acetate succinate (HPMCAS) at both 25% and 50% of compound loading. Solid dispersions were prepared by a solvent evaporation method, using spray-drying followed by secondary drying in a low-temperature convection oven. Dispersions containing 10 mg, 20 mg, 40 mg, 80 mg, 120 mg and 160 mg, of the compound, were prepared into capsules and administered to patients Pharmacokinetics (PK) of the compound of Formula (1) as single agent were characterized in patients with hematologic malignancies, including a population pharmacokinetic modeling. A multicenter, dose escalation study in cohorts of 3 to 6 patients with acute leukemia or other hematologic malignancies was performed with a dose escalation step followed by expansion cohorts at the recommended dose. Patients received oral compound of Formula (1) from 10 to 160 mg according to different schedules. Schedules included once a day dosing with 21-day cycles, acute leukemia (AL); 14 days on/7 days off, hematologic malignancies (OHM). Continuous PK blood samples from 7 time points over 24 hours post-administration were collected on day 1 of cycle 1 for the first 3 patients per DL, and 5 time points over 8 hours post-administration for other patients at this DL. Residual sampling before administration on days 8, 15 and 22 was performed in all patients.

Compound plasma concentrations were measured using validated ultra-performance liquid chromatography with tandem mass spectrometry detection with a concentration range 1-250 ng/mL, with a small plasma volume for analysis (50 μL). Analyses and population PK (PPK) modeling were performed with the nonlinear mixed effect modeling software program Monolix version 4.3. The following parameters were calculated: absorption constant (Ka); apparent distribution volume (V/F); apparent clearance (CL/F) and lean body mass (LBM; calculated considering patient sex, weight and height). Parameters were estimated by computing the maximum likelihood estimator without linearization using the stochastic approximation expectation maximization (SAEM) algorithm combined with a Markov Chain Monte Carlo (MCMC) procedure. The number of MCMC chains was fixed at 10 for all estimations. A constant error model was used to describe residual and Between-Subject Variability (BSV, η) were ascribed to an exponential model. In the absence of intravenous data, the initial bioavailability (F) was set to 1, meaning that clearance (CL) and volume of distribution (V) should be interpreted as the apparent clearance (CL/F) and apparent volume of distribution (V/F), respectively.

Eighty-five patients enrolled and treated and randomized to six dose levels (10, 20, 40, 80, 120 and 160 mg) once-a-day and 40 mg twice-a-day. Among them, 81 patients with 630 plasma concentrations (607+23 BLQ) were evaluable for PK assessment. A 1-compartment open model adequately described the total compound concentration-time curve. The PPK parameters obtained for the structural model were Ka=0.74 h⁻¹ (12%); V/F=71.7 L (6.0%) and CL/F=8.45 L/h (5.0%). The best correlation between compound AUC values and dose was observed from 10 to 120 mg dose levels (R²=0.71). The absorption phase was linear and Tmax was between 1 and 4 h. Mean elimination half-life of compound for all patients was 5.8 h (±1.1). In the PPK study, the best descriptive model was obtained when LBM was considered in the analysis. A correlation between CL/F and V/F was also observed for compound.

A total of 81 patients (52 male, 29 female) were evaluable for PK (40 AL patients, 41 OHM patients), with a total of 633 plasma samples (including 24 below the limit of quantification) available for analysis. Results are shown in Table 1.

TABLE 1
Compound of Formula (1) PK parameters (mean +/− SD) for each dose level derived from EBE

| | Level dose | | | | | | |
|---|---|---|---|---|---|---|---|
| | 10 mg QD | 20 mg QD | 40 mg QD | 80 mg QD | 120 mg QD | 160 mg QD | 40 mg BID |
| | (n = 8) | (n = 6) | (n = 8) | (n = 11) | (n = 30) | (n = 5) | (n = 13)** |
| Cₘₐₓ (μg/L)*** | 131 +/− | 275 +/− | 627 +/− | 1147 +/− | 1474 +/− | 1292 +/− | 550 +/− |
| [mean +/− SD] | 53 | 95 | 307 | 644 | 534 | 511 | 190 |
| AUC_{0-24 hr} | 1187 +/− | 2932 +/− | 5496 +/− | 12466 +/− | 17208 +/− | 13921 +/− | 13111 +/− |
| (μg/L*h) | 125 | 594 | 1222 | 5960 | 7001 | 3235 | 3991** |
| [mean +/− SD] | | | | | | | |
| T_{1/2} (h) | 5.84 +/− | 5.63 +/− | 5.33 +/− | 5.51 +/− | 5.91 +/− | 5.48 +/− | 6.22 +/− |
| [mean +/− SD] | 0.28 | 0.28 | 0.80 | 0.65 | 1.50 | 0.76 | 1.00 |
| V (L) | 72.1 +/− | 57.6 +/− | 58.65 +/− | 71.14 +/− | 63.32 +/− | 96.66 +/− | 57.46 +/− |
| [mean +/− SD] | 11.5 | 11.7 | 14.7 | 28.2 | 15.5 | 16.4 | 12.8 |
| CL (L/h) | 8.53 +/− | 7.06 +/− | 7.66 +/− | 7.83 +/− | 7.81 +/− | 12.10 +/− | 6.57 +/− |
| [mean +/− SD] | 0.95 | 1.19 | 1.72 | 3.28 | 2.35 | 2.65 | 1.65 |
| Cₘᵢₙ (μg/L)*,*** | 22.5 +/− | 50.5 +/− | 47.7 +/− | 113.7 +/− | 325.4 +/− | 215.0 +/− | 351.16 +/− |
| [mean +/− SD] | 11.9 | 7.6 | 16.0 | 56.8 | 249.7 | 164.1 | 183 |

*Observed values;
**Values doubled to mimic 80 mg (2 × 40 mg);
***Predose at Day 8.
SD means standard deviation.
EBE means bempirical Bayesian estimates.

### Example 2

Doses of the compound of Formula (1) were administered to patients having leukemia, hematologic malignancies, diffuse large B-cell lymphoma, other lymphomas or multiple myeloma, and dose limiting toxicity and objective responses were observed.

TABLE 2
Compound of Formula (1) Dose Limiting Toxicity

| | | | | Older Adult Hematologic | |
|---|---|---|---|---|---|
| | | Acute Leukemia | | Malignancies | |
| Dose (mg) | | patients | observed toxicity | patients | observed toxicity |
| 20 | QD | 6 | 0 | 6 | 0 |
| 40 | QD | 4 | 0 | 3 | 0 |
| 80 | QD | 4 | 0 | 7 | 2 |
| 40 | BID | 6 | 0 | 6 | 5 |
| 120 | QD | 11 | 0 | 18 | 10 |
| 160 | QD | 5 | 2 | — | — |

QD mean once per day.
BID means twice per day

Compound of Formula (1) dose-limiting toxicity was evaluated in patients with acute leukemia and hematologic malignancies. In this study, in patients with acute leukemia in this study, dose-limiting toxicity was observed in 2 patients at the 160 mg per day dose (diarrhea and fatigue/anorexia). In patients with hematologic malignancies, dose-limiting toxicity was observed in 2 patients at the 80 mg per day dose (thrombocytopenia), 5 patients at the 40 mg twice per day dose (thrombocytopenia), and 10 patients at the 120 mg per day dose (thrombocytopenia, hypo Na, neutropenia, fatigue, diarrhea, and mucositis).

Compound of Formula (1) once-a-day doses of 10, 20, 40, 80, 120 and 160 mg and twice-a-day doses of 40 mg were administered to patients with leukemia, diffuse large B-cell lymphoma, other lymphomas or multiple myeloma. In this study, objective responses were not observed in patients receiving 10 or 20 mg per day.

1. A method for treating patients with cancer comprising administering to the patient a safe and effective dose of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, wherein the dose is between about 40 mg once per per day and about 120 mg once per per day.

2. A method of claim 1, wherein the dose is about 40 mg once per per day, about 80 mg once per day or about 120 mg once per per day.

3. A method of claim 2, wherein the dose is about 40 mg once per day.

4. A method of claim 2, wherein the dose is about 80 mg once per day.

5. A method of claim 2, wherein the dose is about 120 mg once per day.

6. A method of claim 2, wherein the cancer is acute leukemia, older adult hematologic malignancy, lymphoma or multiple myeloma.

7. A method of claim 6, wherein the lymphoma is diffuse large B-cell lymphoma.

8. A pharmaceutical composition comprising between about 40 mg and about 120 mg (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, and a pharmaceutically acceptable carrier.

9. A pharmaceutical composition of claim 8 comprising about 40 mg (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, and a pharmaceutically acceptable carrier.

10. A pharmaceutical composition of claim 8 comprising about 80 mg (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, and a pharmaceutically acceptable carrier.

11. A pharmaceutical composition of claim 8 comprising about 120 mg (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, and a pharmaceutically acceptable carrier.

12. A method for treating patients with cancer comprising administering to the patient a dose of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide or a pharmaceutically acceptable salt or hydrate thereof, wherein the dose provides safe and effective drug exposure.

13. A method of claim 12, wherein the dose is between about 40 mg once per per day and about 120 mg once per per day.

14. A method of claim 13, wherein the dose is about 40 mg once per per day, about 80 mg once per day or about 120 mg once per per day.

15. A method of claim 14, wherein the dose is about 40 mg once per day.

16. A method of claim 14, wherein the dose is about 80 mg once per day.

17. A method of claim 14, wherein the dose is about 120 mg once per day.

18. A method of claim 14, wherein the cancer is acute leukemia, older adult hematologic malignancy, lymphoma or multiple myeloma.

19. A method of claim 18, wherein the lymphoma is diffuse large B-cell lymphoma.

20. A method of claim 12, wherein drug exposure corresponds to AUC₀₋₂₄ of between about 3000 to about 20000 μg/L*h.

21. A method of claim 20, wherein drug exposure corresponds to AUC₀₋₂₄ of between about 5000 to about 18000 μg/L*h.

22. A method of claim 12, wherein drug exposure corresponds to T_{1/2} of between about 5 and about 6 hours.

23. A method of claim 12, wherein drug exposure corresponds to Cₘₐₓ of between about 500 and about 1500 μg/L.

24. A method of claim 12, wherein drug exposure corresponds to Cₘₐₓ of between about 600 and about 1500 μg/L.
